(19) United States
(12) Patent Application Publication
(10) Pub. No.: US 2019/0350496 A1
(43) Pub. Date: Nov. 21, 2019

(54) BODY PART MOTION ANALYSIS USING KINEMATICS

(71) Applicant: Figur8, Inc., Boston, MA (US)

(72) Inventors: Nan-Wei Gong, Cambridge, MA (US); Donna Susan Scarborough, Hingham, MA (US)

(73) Assignee: Figur8, Inc., Boston, MA (US)

(21) Appl. No.: 16/529,851

(22) Filed: Aug. 2, 2019

Related U.S. Application Data
(63) Continuation-in-part of application No. 15/875,311, filed on Jan. 19, 2018.
(60) Provisional application No. 62/714,241, filed on Aug. 3, 2018.
(60) Provisional application No. 62/742,222, filed on Oct. 5, 2018.
(60) Provisional application No. 62/821,071, filed on Mar. 20, 2019.
(60) Provisional application No. 62/448,525, filed on Jan. 20, 2017.
(60) Provisional application No. 62/464,443, filed on Feb. 28, 2017.
(60) Provisional application No. 62/513,746, filed on Jun. 1, 2017.

(51) Int. Cl. A61B 5/11; A61B 5/00

(52) U.S. Cl. CPC A61B 5/1107; A61B 5/1118; A41D 1/002; A61B 2560/0412; A61B 5/6804

(57) ABSTRACT

Disclosed embodiments describe techniques for body part motion analysis using kinematics. Two or more sensors, which include stretch sensors or inertial sensors, are attached to a body part of an individual. The two or more sensors enable collection of motion data of the body part. Data is collected from the two or more sensors, where the two or more sensors provide electrical information based on a micro-expression of movement of the body part. One or more processors are used for analyzing the electrical information from the two or more sensors to generate a kinematic phase pattern. The kinematic phase pattern is rendered as part of a kinematic sequence, where the rendering is displayed visually. The rendering includes an animation and enables additional kinematic analysis. The two or more sensors capture two or more modalities of body part motion.

### RELATED APPLICATIONS

This application claims the benefit of U.S. provisional applications “Body Part Motion Analysis Using Kinematics” Ser. No. 62/714,241, filed Aug. 3, 2018, “Wearable Sensors with Ergonomic Assessment Metric Usage” Ser. No. 62/742,222, filed Oct. 5, 2018, and “Human Body Mounted Sensors with Mapping and Motion Analysis” Ser. No. 62/821,071, filed Mar. 20, 2019.

This application is also a continuation-in-part of U.S. patent application “Body Part Deformation Analysis Using Wearable Body Sensors” Ser. No. 15/875,311, filed Jan. 19, 2018, which claims the benefit of U.S. provisional patent applications “Body Part Deformation Analysis with Wearable Body Sensors” Ser. No. 62/448,525, filed Jan. 20, 2017, “Body Part Deformation Analysis using Wearable Body Sensors” Ser. No. 62/464,443, filed Feb. 28, 2017, and “Body Part Motion Analysis with Wearable Sensors” Ser. No. 62/513,746, filed Jun. 1, 2017.

Each of the foregoing applications is hereby incorporated by reference in its entirety.

### FIELD OF ART

This application relates generally to motion analysis, and more particularly to body part motion analysis using kinematics.

### BACKGROUND

The detection and measurement of motion and deformation of a given shape are of keen interest to a variety of research, computational, manufacturing, and other fields. The accurate measurement of the motion and the deformation of the shape directly applies to machine vision, industrial automation, scientific biomechanics research, medical treatment, and three-dimensional animation, among many others. The types of shapes that are measured include objects of interest, manufactured parts, body parts, etc. The measurements can be used for object differentiation, where the object differentiation is based on material, size, shape, location, or cost, among many other parameters. When the shape being measured is a portion of a body such as the human body, then measurement of the shape has further applications in industries such as healthcare, sports, fashion, or 3D animation for entertainment and gaming. Accurate shape measurement can be used to obtain critical data such as personal medical information and can be used to design proper medical treatments. Proper medical treatments are essential for comfort, safety, and therapeutic outcomes for an individual.

In a clinical setting, accurate and precise human body measurements are difficult to obtain. To start with, consider a relatively simple, static, volumetric body part measurement, such as measuring the volume of fluid buildup in a limb caused by lymphedema. This is typically a manual process where a tape measure is often used by a clinical professional to make body measurements. First the limb is marked along a longitudinal axis using the tape measure and a marking pen. An appropriate gradation, say every 1 cm, is marked. Next, a transverse circumference is measured at every gradation and recorded. The transverse circumferential measurements are repeated along the desired length of the limb. At a subsequent clinical visit, say one week or one month later, the measurements are taken again. Total limb volume V can be approximated by assuming a step-wise linear series of cylindrical disks. The volume V can be expressed as the area A of each transverse cross-section (where A=C²/4π, and where C is the measured circumference) times the height h of each gradation, and then summing all of the cylindrical disk volumes into the total volume. In this way, lymphedema progression and/or treatment effectiveness can be monitored.

Unfortunately, even though this is a relatively simple example involving a static measurement of a non-moving body part, the typical clinical approach is fraught with inconsistencies and opportunities for human error. A different person may be making the measurements. Inconsistent pressure may be applied when measuring the circumference. The tip of the marking pen can be several mm wide. Subtle limb shape changes, whether related to lymphedema or not, may greatly affect the accuracy of the estimated volumetric model calculation. Many such difficulties exist for making even this relatively simple static, body part measurement.

While making static body part measurements is very difficult, it is even more difficult to measure moving body parts, such as a joint. Body part joint movement is three-dimensional, and the movement happens in real-time, that is, non-static. By necessity, the body part joint is moving when a measurement needs to be taken. Body part joint measurements can involve different deformations along multiple axes. Multiple measurements of a repetitive motion may be required. Measurements may need to be made while the body part is under a load condition or under nominal conditions. All of these variables present an additional layer of variation that makes measurement difficult. Added to all that complexity is the fact that body part joints are connected to other body part joints, which further complicates measurement and analysis of shape motion and deformation. Accordingly, a great need exists to be able to accurately measure and analyze body part motion.

### SUMMARY

The successful analysis of the motion of a body part is critically linked to the accurate measurement of the motion. The analysis of the motion can be used for medical or injury diagnostics, for measuring medical treatment efficacy, or for sport performance enhancement. Techniques are disclosed for body part motion analysis using kinematics. At least two sensors, such as stretch sensors or inertial measurement units (IMU), are applied to a body part of an individual. A stretch sensor changes electrical characteristics as the electroactive polymer comprising the sensor is stretched. An IMU changes electrical characteristics as the IMU accelerates, rotates, or changes position. The sensors are attachable to a body part. Tape, wrap, or a garment can be applied to the body part, and the sensors can be attached to the tape, wrap, or garment using hooks. The tape, for example, can be a specialized tape such as a physical therapy tape, surgical tape, therapeutic kinesiology tape, and so on. One or more strips of tape can be attached to the body part. The one or more strips of tape can be attached in various configurations. The body part can include one or more of a knee, shoulder, elbow, wrist, hand, finger, thumb, ankle, foot, toe, hip, torso, spine, arm, leg, neck, jaw, head, or back. Data including changes in electrical information is collected from the two or more sensors, where the changes in electrical information are due to motion of the body part. An at least third sensor can be applied to the body part to determine and analyze motion between symmetrical body parts. A communication unit, coupled to the sensors, provides information from the sensors based on the changes in electrical characteristics. The communication unit can provide information using wired and wireless techniques. A receiving unit receives the information provided by the communication unit. The received information is analyzed to generate a kinematic phase pattern. The kinematic phase pattern is rendered as part of a kinematic sequence, and the rendering is displayed visually. The rendering can include an animation of the body part.

A processor-implemented method for motion analysis is disclosed comprising: attaching two or more sensors to a body part of an individual, wherein the two or more sensors enable collection of motion data of the body part, and wherein the two or more sensors include at least one inertial measurement unit (IMU) and at least one sensor determining muscle activation; collecting data from the two or more sensors, wherein the two or more sensors provide electrical information based on a micro-expression of movement of the body part; and analyzing, using one or more processors, the electrical information from the two or more sensors to generate a kinematic phase pattern. The kinematic phase pattern is rendered as part of a kinematic sequence, and the rendering is displayed visually. In embodiments, the two or more sensors comprise one or more integrated sensors, and the one or more integrated sensors comprise stretch sensors. In embodiments, the two or more sensors comprise a network of sensors. In embodiments, the two or more sensors capture two or more modalities of body part motion. And in embodiments, at least one of the two or more sensors comprises a bending sensor.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for body part motion analysis using kinematics.

FIG. 2 is a flow diagram for calculating kinematic summation and distribution ratio.

FIG. 3 shows sensor configuration.

FIG. 4 illustrates sensor placement.

FIG. 5A shows shoulder motion.

FIG. 5B shows data collected from shoulder.

FIG. 6 illustrates a plot of jump data.

FIG. 7 shows a block diagram for a kinematic phase pattern from muscle data.

FIG. 8A illustrates a dashboard showing jump and recording.

FIG. 8B illustrates a dashboard showing a jump report.

FIG. 9A shows example lower body sensor locations.

FIG. 9B shows example upper body sensor locations.

FIG. 10 is a system diagram for body part motion analysis using kinematics.

### DETAILED DESCRIPTION

Techniques are disclosed for body part motion analysis using kinematics. Two or more wearable sensors can be attached to a body part of an individual. The wearable sensors comprise stretch sensors for evaluating motion of a portion of the body, and inertial measurement units (IMU) for measuring acceleration, rotation, and position. The wearable sensors can be attached to a fabric which can be attached to a body part. The fabric can include tape, a woven fabric, a knitted fabric, a garment, etc. The tape can be a specialized tape such as a physical therapy tape, surgical tape, therapeutic kinesiology tape, and so on. The sensors can be used to measure various parameters relating to movement of the body part. The measurement of body part can be used to perform symmetry evaluation, to evaluate a similar body part, to evaluate symmetrical operation of similar body parts, to perform micro-expression movement evaluations, to evaluate angle, force and torque, etc. The body part can include one or more of a knee, shoulder, elbow, wrist, hand, finger, thumb, ankle, foot, toe, hip, torso, spine, arm, leg, neck, jaw, head, or back. The first stretch sensor changes electrical characteristics as the first stretch sensor stretches and typically detects linear displacement through stretching. As a sensor stretches, an angle of deformation can be determined for a portion of a body such as a knee, shoulder, or elbow. The electrical information can include changes in capacitance, resistance, impedance, inductance, etc. An electrical component coupled to the stretch sensor collects the changes in electrical characteristics by the stretch sensor based on motion of the body part. The sensor collects changes in capacitance, resistance, impedance, inductance, and so on. Information from the sensor can be augmented with the inertial measurement unit (IMU) information. A communication unit, coupled to the sensor, provides electrical information from the sensor on the changes in electrical characteristics by the stretch sensor. The electrical information provided by the communication unit is received by a receiving unit, separate from the first electroactive polymer, the sensor, and the communication unit. The electrical information is analyzed to generate a kinematic phase pattern. The kinematic phase pattern can be rendered as part of a kinematic sequence. The kinematic phase sequence can be rendered and displayed visually. A rendering on the display shows motion of the body part based on the information that was received by the receiving unit. The display shows an animation of the body part based on the motion of the body part based on the changes in electrical characteristics by the first stretch sensor. In embodiments, the body part is displayed in a context of an overall body of which the body part is a portion.

Traditional inertial measurement unit-based systems attempt to infer the “absolute” location of a certain point of interest by integrating the acceleration reading in a 3D space. However, the accuracy of such an approach is limited by the sampling rate and the accuracy of the on-board accelerometer. One problem that is frequently encountered by all of these IMU-based solutions is called drift. Drift is the error (here in location distance) between the actual location of an object versus the location that is calculated/observed by the IMU reading. The drift error results over time from the accumulative error, which is based on the calculation. The approach taken here is based on measuring joint angle, where the angle is linear to the displacement reading of the stretch sensor. This approach does not suffer from the accumulative error. Body movement, or 3D motion of a body part, such as a hand gesture, can be accurately represented in a 3D space over time.

Disclosed techniques address sensing of body part motion and motion analysis using kinematics. In embodiments, tape such as physical therapy tape, therapeutic kinesiology tape, surgical tape, etc. can be applied to a body part. In other embodiments, the body part can be wrapped, placed in a garment, etc. The body part can include one or more of a knee, shoulder, elbow, wrist, hand, finger, thumb, ankle, foot, toe, or hip, or other body parts such as torso, spine, arm, leg, neck, jaw, head, or back. In embodiments, the tape can be applied to symmetrical body parts such as left shoulder and right shoulder, left hip and right hip, etc. Two or more stretch sensors can be applied to the tape that is applied to a body part. The attaching of the two or more sensors to the tape, wrap, garment, etc., can be accomplished using hooks, a hook and loop technique, fasteners, clips, bands, and so on. The two or more sensors that can be applied can provide electrical information which, when analyzed, can provide an inferred joint angle movement based on absolute linear displacement of the one or more stretch sensors. The absolute linear displacement information can be augmented with information collected by an inertial measurement unit (IMU). The inertial measurement unit can include a six-axis or nine-axis IMU. The six-axis IMU can include a gyroscope for three axes, and an accelerometer for three axes. The nine-axis IMU can include a gyroscope for three axes, an accelerometer for three axes, and a magnetometer for an additional three axes. The addition of the magnetometer in the nine-axis IMU can improve accuracy. While the gyroscope and accelerometer can provide information about acceleration and rotation, their accuracy to measure location decreases over time due to drift. The information provided by the magnetometer can provide additional absolute direction sensing. The magnetometer measurements can be used to compensate for the drift over a time interval.

Techniques for motion analysis can be used for body part motion analysis. The body part analysis can include tracking symmetrical body parts as the body parts are moved. The movement of the body parts can be related to tracking body part motion, body part diagnosis, body part test, body part therapy, and so on. The body part motion analysis can include acceleration and orientation information. The acceleration and orientation information relating to a body part can be collected by a six-axis or a nine-axis inertial measurement unit (IMU). The six-axis IMU can include acceleration and rotation, and the nine-axis IMU can include acceleration, rotation, and absolute direction information. A stretch sensor can be used to determine motion. In embodiments, the stretch sensor prevents an error due to drift and can be used to determine an angle and sagittal plane flexion and/or extension motion of a body part. The stretch sensor can include an electroactive polymer. More than one stretch sensor can be used. An additional stretch sensor can be attached at a right angle with respect to the first stretch sensor. The addition of the additional stretch sensor can be used to determine muscle function. The muscle function can include muscle stretch, muscle angle, muscle bulge, and so on.

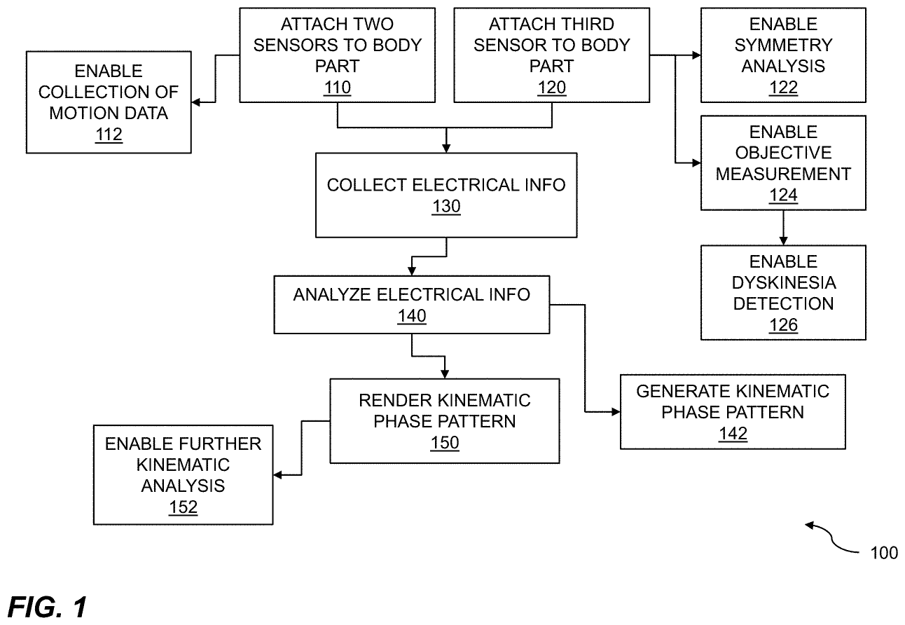

FIG. 1 is a flow diagram for body part motion analysis using kinematics. Sensors are used to collect motion data from a body part of an individual. The motion data includes electrical information related to movement of the body part. Analysis of the electrical information reveals a kinetic phase pattern which can be part of a kinetic sequence. The kinetic sequence, which includes movement and energy information about the body part, can be used for medical diagnosis, treatment of injury, sport performance improvement, and so on. The flow 100 includes attaching two or more sensors to a body part 110 of an individual. The body part can include a muscle such as a bicep, triceps, or quadricep; or a joint such as a shoulder, elbow, wrist, hip, knee, or ankle. The attaching can be accomplished using hooks which can attach to tape, a wrap, a garment, etc. The tape can include physical therapy tape and therapeutic kinesiology tape, a woven material, etc. The two or more sensors enable collection of motion data 112 of the body part. The two or more sensors can be the same type of sensor or different types of sensors. In embodiments, the two or more sensors can include stretch sensors. A stretch sensor can measure stretch using a variety of materials and techniques. In embodiments, the sensor can include an electroactive polymer. An electroactive polymer is typically defined as a polymer that changes size or shape when activated by an electrical signal or field. However, other applications of electroactive polymers include the change of electrical characteristics of the electroactive polymer as it is stretched or deformed. The electrical characteristics that can change can include resistance, capacitance, inductance, etc. Other types of sensors can also be used.

In embodiments, the two or more sensors can include inertial measurement units (IMU). An IMU can include a variety of measurement components such as one or more of an accelerometer, a gyroscope, and a magnetometer. The IMU can measure acceleration, rotation, position, and so on. The flow 100 further includes attaching at least a third sensor to the body part 120. Data provided by the at least third sensor can be used to confirm data collected from the other sensors or can provide additional information. In embodiments, the at least third sensor can enable body part symmetry analysis 122. Body part symmetry analysis can be used to measure the range of motion, the stretch, and other characteristics related to the body parts. For example, the body part symmetry analysis can be used to measure motion of the left shoulder and the right shoulder of the individual. In embodiments, the at least a third sensor enables an objective measurement 124 of scapular movement. In embodiments, the objective measurement 124 of scapular movement enables dyskinesia detection 126 of the scapula.

The flow 100 includes collecting data from the two or more sensors, where the two or more sensors provide electrical information 130 based on a micro-expression of movement of the body part. The electrical information can be based on a DC signal, an AC signal, pulses, and so on. The electrical information can include values related to resistance, capacitance, inductance, etc. The electrical information that is collected from the two or more sensors can be held or stored within the two or more sensors, transmitted to a receiving component, etc. The transmitting of the electrical information can be accomplished using one or more wireless techniques including Wi-Fi, Bluetooth198 , Zigbee™, near-field communication (NFC), and the like. The transmitting of the electrical information can include continuous transmission, burst transmission, intermittent transmissions, etc. The flow 100 includes analyzing, using one or more processors, the electrical information from the two or more sensors 140 to generate a kinematic phase pattern 142. A kinematic sequence can be understood as the timing of angular velocities generated across related body parts or segments. The angular velocities can be the peak angular velocities. Movement patterns can be captured by assessing the timing of peak angular velocities. The comparison of the proportion of the peak generated velocities (or other metrics of torques, forces, muscle output, etc.) during a pattern of movement can be a better quantification of the pattern of movement than other comparisons, especially when the comparison is performed against a library of collected kinematic sequences. Using this method, the contribution of motion across each joint to the total pattern in magnitude can be defined using the two or more sensors. Both “open chain” patterns, where a distal segment is free (throwing or reaching), as well as “closed chained” patterns, where the distal segment is against a surface (pushing an object), can be evaluated.

In embodiments, the one or more processors can be coupled to the two or more sensors or can be beyond the two or more sensors. The two or more processors can include a smart device, a smartphone, PDA, laptop, a local server or blade server, a remote server, cloud-based server, etc. In embodiments, the electrical information from the two or more servers can be analyzed on a tablet. In embodiments, the two or more sensors comprise one or more integrated sensors. For example, an integrated sensor may include a sensor to detect muscle activation and an IMU to detect muscle movement based on spatial positioning, acceleration, velocity, and so on. In embodiments, the one or more integrated sensors comprise stretch sensors, as will be discussed later. In embodiments, the two or more sensors comprise a network of sensors. The network of sensors can be attached to a single body for multiple measurements of complex movement of that body or body part. The network of sensors can be attached to more than one body. For example, several bodies can have two or more sensors attached to the same body part on each body. Comparative muscle movement on each of the bodies is thereby enabled. In embodiments, the two or more sensors capture two or more modalities of body part motion. The modalities can include muscle activation, or contraction, and muscle movement in three-dimensions. The modalities can be assessed using a stretch sensor and an IMU, although other sensors for capturing modalities are possible.

The kinematic phases can include a phase pattern. A kinematic phase pattern can refer to a cycle of movements of a body part, where such movements can be related to walking or running, raising and lowering arms, engaging in a sport, and so on. A variety of phases can be described. A phase can include a stance-phase. A stance-phase can include a double stance for an activity such as walking. The double stance can include a heel strike, a mid-stance where the legs are vertical, a toe-off, and so on. The double stance such as walking or running can include a movement of a center of mass related to the body of the individual. In the case of walking, the center of mass of the individual will appear to rise at mid-stand and fall for heel-strike or toe-off. Another phase can include a swing-phase. In a swing phase, a leg to be moved forward undergoes knee-flexion to raise the leg before being swung forward.

The flow 100 includes rendering the kinematic phase pattern as part of a kinematic sequence 150. A kinematic sequence can be used to describe a sequence of movements of a body part. Further, the kinematic sequence can describe how the sequence of movements can be used to transfer energy throughout the body. A kinematic sequence can describe a jump or a start for a running sprint or a swimming event, the swing of a piece of sports equipment, etc. In embodiments, the swing of a piece of sports equipment can include a swing of a golf club, a tennis racquet, a ball bat such as a baseball or cricket back, a lacrosse stick, a fly-fishing rod, and so on. The rendering can be displayed visually. The visual rendering can be displayed on an electronic display coupled to the one or more processors, on a computing device, etc. The electronic display can be on a smartphone, a laptop computer, a tablet computer, a personal digital assistant (PDA), a television monitor, a projector, or any other type of electronic device. In embodiments, the rendering includes an animation of the body part. The animation of the body part can be based on a stock animation that can illustrate joint movement or muscular activity, an animation based on the individual, and so on. In embodiments, the rendering enables additional kinematic analysis 152. The additional kinematic analysis can be performed by the one or more processors, a computing device, a human viewer of the rendering, etc.

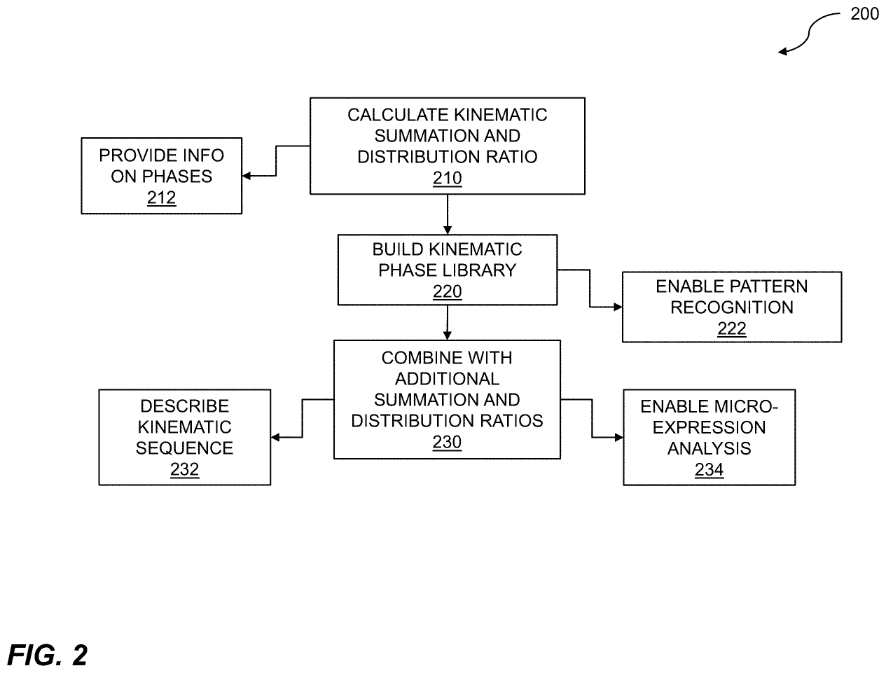

FIG. 2 is a flow diagram for calculating kinematic summation and distribution ratio. A kinematic summation and distribution ratio can be calculated based on the micro-expression of movement of a body part. The kinematic summation and distribution ratio can be used for body part motion analysis using kinematics. Two or more sensors are attached to a body part of an individual, where the sensors enable collection of motion data. Sensor data is collected, where the data includes electrical information based on a micro-expression of movement of the body part. The electrical information is analyzed to generate a kinematic phase pattern. The kinematic phase pattern is rendered as part of a kinematic sequence.

The flow 200 includes calculating a kinematic summation and distribution ratio 210 based on the micro-expression of movement of the body part. The kinematic summation and distribution can be calculated using one or more processors. The calculating can be used to determine information such as position, rotation, movement, acceleration, and so on, related to the body part. In embodiments, the calculating provides information on kinematic phases 212. The kinematic phases can include phase patterns, where a kinematic phase pattern can refer to a cycle of movements of a body part, where such movements can be related to walking or running, raising and lowering arms, and so on. A phase can include a stance-phase. A stand-phase can include a double stance from walking. The double stance can include a heel strike, a mid-stance where the legs are vertical, a toe-off, and so on. The double stance can include a movement of a center of mass. In the case of walking, an individual's head will appear to rise at mid-stand and fall for heel-strike or toe-off. Another phase can include a swing-phase. In a swing phase, a leg to be moved forward undergoes knee-flexion before being swung forward.

The flow 200 includes using the information on kinematic phases to build a kinematic phase library 220. A kinematic phase library can be used to collect, aggregate, store, etc., kinematic phase patterns. The kinematic phase patterns can result from analysis of the electrical information collected from the two or more sensors attached to the body part of the individual. The kinematic phase library can include data collected from other individuals. The data collected from the other individuals can represent populations of individuals, where the populations can include normal or healthy populations, injured populations, and so on. The data contained in the kinematic phase library can be compared to data from the individual to determine an injury, to measure progress of strengthening or healing, etc. The flow 200 can include using the kinematic phase library to enable pattern recognition 222 for information on kinematic phases obtained from the calculating. The pattern recognition can be used to recognize a kinematic phase, to measure a kinematic phase, to compare a kinematic phase to a “standard”, a “normal”, or a previous measurement obtained from the individual, and the like.

The flow 200 further includes combining the kinematic summation and distribution ratio with one or more additional kinematic summation and distribution ratios 230 for a segment of a related body part. The segment of a related body part can include muscles or joints adjacent to the body part, such as an elbow or a wrist adjacent to a shoulder to which the two or more sensors were attached. The segment of a related body part can include a symmetrical body part such as left or right shoulder, left or right hip, and so on. In embodiments, the combining can describe a kinematic sequence 232. A kinematic sequence can be used to describe a sequence of movements of a body part and how that sequence of movements can be used to transfer energy. A kinematic sequence can describe a jump or a launch from a starting stance for a sprint or an individual medley swimming event, a swing of a golf club or a tennis racquet, arm movement of a baseball pitcher, and so on. In embodiments, the combining can enable micro-expression analysis 234 of the individual. The micro-expression analysis can determine specific motions, paths of travel, velocity, acceleration, etc., related to the body part. The micro-expression analysis can be applied to body part performance, therapies, and so on. In embodiments, the micro-expression analysis of the individual can used for sport performance enhancement. The sport performance enhancement can be used to optimize the swing of a golf club, tennis racquet, lacrosse stick, baseball or cricket bat, and the like. In further embodiments, the micro-expression analysis of the individual can be used for medical treatment. The micro-expression analysis can be used to design a rehabilitation therapy, to measure progress toward a rehabilitation goal, etc. In embodiments, micro-expression analysis of the individual can be used for medical diagnostics. The diagnostic can include excessive motion of a joint, unbalance movement of symmetrical joints, etc. In embodiments, the micro-expression analysis can enable an objective measurement of scapular movement and dyskinesia. In some instances, the manner in which an individual moves a particular body part may increase a risk of injuring the body part. In embodiments, the micro-expression analysis of the individual is used for injury risk analysis. An injury risk analysis can indicate that further tests, measurements, or diagnostics should be performed. In embodiments, the micro-expression analysis of the individual is used for injury diagnostics.

Further to techniques that can calculate kinematic summation and a distribution ratio based on the micro-expression of movement of a body part, other measurement techniques can be used. In embodiments, techniques based on electrical impedance myography can be exploited. Electrical impedance myography (EIM) can be applied to a measurement of an intensity and a velocity of a muscle contraction event, where the measurement can include electrical impedance of the muscle or muscle group. EIM can be a noninvasive technique that can measure the electrical impedance characteristics. The electrical impedance characteristics can be used to determine health of a muscle or group of muscles, such as diagnosing a neuromuscular disease or other medical condition, assessing progression of the disease or condition, etc. The muscle health determination also can be useful for physical therapies, measuring the progress of healing, and so on.

The composition of a muscle or a group of muscles can be altered by the occurrence of disease, as can the microstructure of the muscle or group of muscles. By measuring changes in electrical impedance of the muscle or muscles using EIM, the occurrence of a disease such as a neuromuscular disease can be detected. The measurement of muscle impedance can be represented by a resistance-capacitance (RC) model, where the resistance component can be associated with cellular fluids within the muscles, and the reactance component can be associated with capacitive effects attributable to the cell membranes of the cells within the muscles. The cellular fluid can include extracellular fluid and intracellular fluid. The cell membranes can represent the capacitor dielectric separating the extracellular and intracellular fluids. Since disease can alter, sometimes significantly, the membranes of the cells, the impedance of the calls can also undergo significant impedance changes. Thus, measuring the impedance of the muscle or muscle group over time can determine disease presence, disease progression, atrophy of muscle fibers, etc.

Impedance, such as electrical impedance associated with myography, is based on real components described as resistance, and imaginary components described as reactance. By applying a signal such as a sinusoidal signal to the surface of a muscle or muscle group, and by measuring the amount of time or time delay taken for the signal to pass through the muscle, a phase value can be calculated. By measuring resistance and reactance, and by calculating phase, a muscle disease may be identified. Electrical impedance myography can be impacted by physical characteristics of the patients for whom EIM is being performed. Physical characteristics of the patient can include thickness of the skin, the amount of fat under the skin (subcutaneous fat), and so on. By applying more than one sinusoidal test signal, where the sinusoidal test signals are based on different frequencies, the effects of skin and fat can be on impedance measurements can be reduced. Further, an amount of subcutaneous fat between the skin and the muscle may also be determined. In embodiments, at least one of the two or more sensors comprises an electromyogram sensor.

Other body part movement detection techniques include mechanomyography, which is sometimes called phonomyogram, acoustic myogram, or sound myogram. Mechanomyography relies on sensing muscle activation through resultant stimulation of adjacent media. For example, a muscle twitch can cause a movement of the skin above or near the muscle, which can then result in the movement of air molecules around the skin that can be detected as sound waves. Thus in embodiments, at least one of the two or more sensors comprises a mechanomyogram sensor.

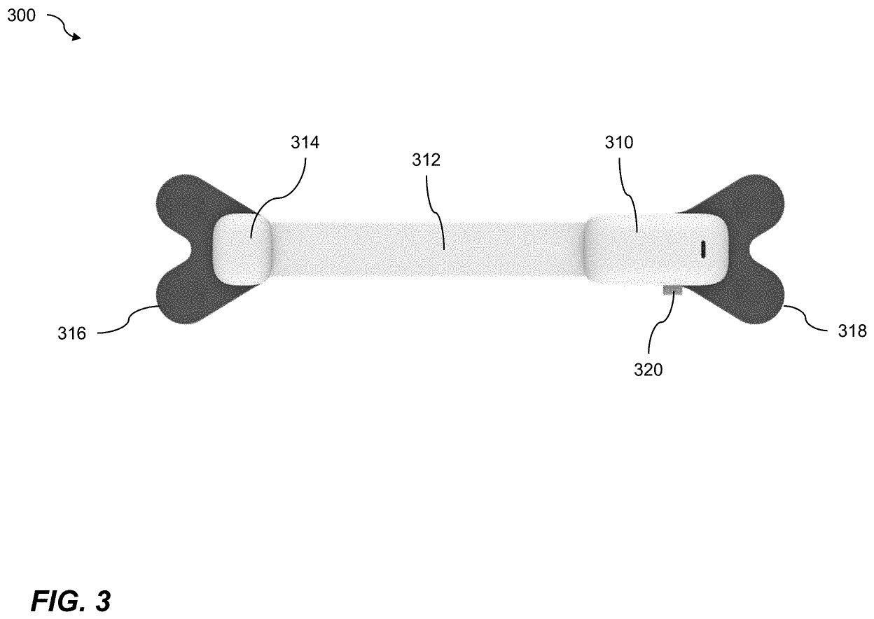

FIG. 3 shows sensor configuration. Two or more sensors can be attached to a body part of an individual and used for body part motion analysis using kinematics. The sensors can include stretch sensors, inertial measurement units (IMU), and so on. An IMU can include one or more of an accelerometer, a gyroscope, and a magnetometer. Data is collected from the sensors, where the sensor data includes electrical information based on a micro-expression of movement of the body part. Processors are used to analyze the electrical information to generate a kinematic phase pattern. The kinematic phase pattern is rendered as part of a kinematic sequence, where the rendering can be displayed visually. The rendering can include an animation of the body part.

A stretch sensor configuration 300 for attachment to a body part is shown. A stretch sensor, an IMU, or other sensors, can be used for body part motion analysis using kinematics. A sensor, such as a stretch sensor or an IMU, changes electrical characteristics as it stretches or moves, respectively. The electrical characteristics can include resistance, capacitance, inductance, reluctance, and so on. The stretching of the stretch sensor can correspond to movement of a body part to which the sensor is attached. Similarly, motion of an IMU can include acceleration, rotation, or position of the body part. A collector or sensor coupled to the stretch sensor to collect changes in electrical characteristics based on motion of the body part. A communication unit provides information from the sensor or collector to a receiving unit. The stretch sensor configuration 300 can comprise an apparatus for attachment to tape on a body part. The sensor configuration can include an electrical component 310. The electrical component 310 can be coupled to a stretch sensor 312 and can collect data relating to changes in electrical characteristics of the stretch sensor 312. The electrical component 310 can include a power source that can provide power to electrical circuits and can drive the stretch sensor 312. The power source and circuitry provide other signals such as sinusoids or pulses at various frequencies, AC or DC voltages, etc., that may be required to operate the sensor. The electrical component can include an electrical characteristic calculation component. The electrical characteristic calculation component can be used to determine stretch, bulge, displacement, and other physical characteristics based on body part motion. The electrical component can include a Bluetooth™, Wi-Fi, Zigbee, or other communication unit which can be used to send collected changes in electrical characteristics of the stretch sensor. While one stretch sensor is shown, other numbers of stretch sensors can be included in a sensor configuration. As stated throughout, additional sensors can be based on IMUs. The electrical component can be coupled to a button 320, switch, or other device for energizing or deenergizing the electrical component.

The stretch sensor 312 can include various materials which can be used to detect or measure stretch. In embodiments, the stretch sensor can include an electroactive polymer. The stretch sensors can be configured in a variety of arrangements such as a t-shape, an offset-t-shape, a w-shape, an x-shape, a spider-shape, and so on. The stretch sensor 312 can be coupled to an anchor 314 for the stretch sensor. The stretch sensor anchor can include hooks, and the anchor can be used to attach the stretch sensor to an anchor 316 and 318. The anchors 316 and 318 can include tape, fabric, wrap, and so on. When tape is used, the tape can be attached to the body part where the first stretch sensor can then be attached to the tape. In embodiments, the tape can include physical therapy tape. In other embodiments, the tape can include therapeutic kinesiology tape.

In other embodiments, the sensor configuration 300 can include a bend sensor. One or more bend sensors can be applied to a body part of an individual and can be used for body part motion analysis. The one or more bend sensors can be used to measure motion of the body part with one or more degrees of freedom. Various techniques can be used to implement a bend sensor such as basing the bend sensor on a compliant capacitive strain sensor. A compliant capacitive strain sensor can comprise a dielectric layer sandwiched between two conducting electrode layers. The dielectric layer and the electrode layers can be can be based on flexible materials, where the flexible materials can include polymers. The flexible materials such as the polymers can include natural rubber, silicone, acrylic, and so on. Since the polymers can typically be insulators, the electrodes of the bend sensor can be formed by introducing conducting particles into the polymers, where the conducting particles can include nickel, carbon black, and the like. In order for the capacitive strain sensor to be applied to the body part, one or more compliant capacitive strain sensors or other strain sensors can be applied to a material such as tape that can be applied to the body part, a fabric that can enwrap the body part, a garment that can be worn on the body part, and so on. In embodiments, at least one of the two or more sensors comprises a bending sensor.

The compliant capacitive strain sensor can measure strain based on the amount of displacement experienced by the strain sensor. The ability of a compliant capacitive strain sensor to measure strain can be limited by the amount of displacement that can be sustained by the strain sensor before the strain sensor is temporarily or permanently damaged. Excessive strain applied to the strain sensor can cause electrical parameters of the strain sensor such as the resistance of the strain sensor to change significantly. The significant change in resistance of the strain sensor can include an “open circuit” (high resistance) resulting from a damaged or destroyed strain sensor.

An application of a sensor, such as the configuration shown, to a body part (e.g. a shoulder) can be used to determine angle measurements for the shoulder. In embodiments, angle measurements can include sagittal plane flexion and extension. In addition to angle measurements for a given body part, muscle function assessment can also be performed. In embodiments, muscle function assessment can include displacement of muscle contraction that can occur during an activity. The activity can include normal physical activity such as yoga and strenuous physical activity such as swimming, rowing, rock climbing, and so on. Peak displacement of a muscle can be based on maximum contraction of key superficial muscle groups. A sensor can be attached to a targeted muscle group, over the location of greatest muscle mass displacement. In addition to peak muscle displacement for muscle function determination, an amount of time required to reach peak muscle contraction can be recorded. Other sensors can be applied to shoulder measurements. In embodiments, the inertial measurement unit (IMU) can be used to track acceleration and orientation of a body part such as a shoulder. Based on measurements collected from the IMU, intersegmental movement can provide information on movement patterns across anatomical joints. The information based on the intersegmental movement provides information on a fluidity of movement and a quality of motion. This information can provide side to side comparison of movement of the anatomical joints for healthy populations in contrast with injured populations.

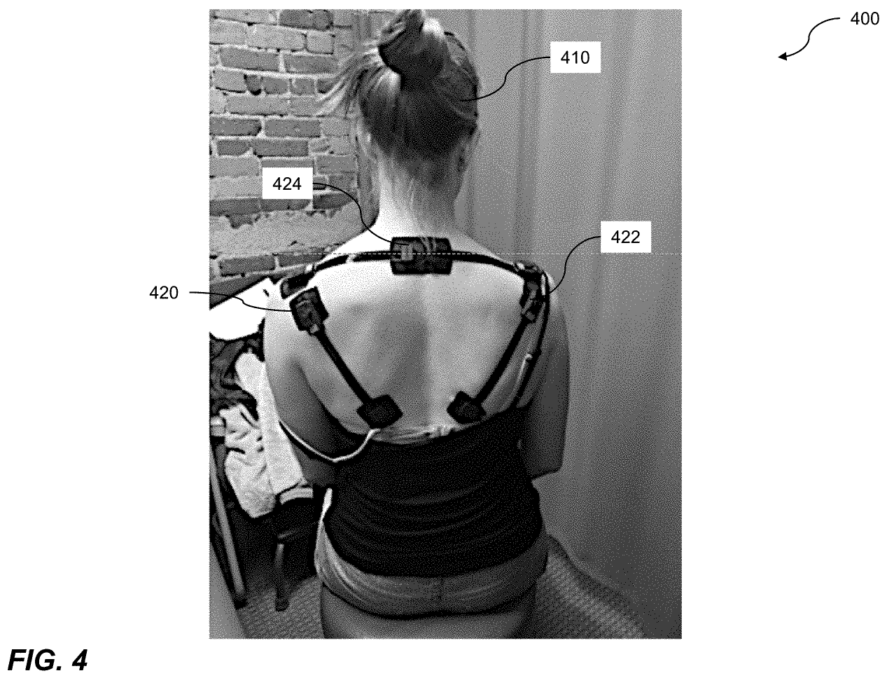

FIG. 4 shows sensor placement. Sensors can be placed on a body part of an individual, where the sensors can include stretch sensors or inertial measurement units (IMU). The sensors can enable collection of motion data of the body part. Data is collected from the sensors, where the data includes electrical information based on a micro-expression of movement of the body part. The electrical information is analyzed to generate a kinematic phase pattern, where the kinematic phase pattern is rendered as part of a kinematic sequence. Placement 400 of sensors on an individual 410 is shown. The sensors, which can include two or more sensors, can be placed on a body part such as hips, knees, ankles, wrists, elbows, or shoulders, as shown. Further embodiments include attaching at least a third sensor to the body part. The third sensor can include a stretch sensor, an IMU, or both. In embodiments, the at least a third sensor can enable body part symmetry analysis. In the figure, three sensors are shown. A first sensor 420 can be attached to the left shoulder of the individual, a second 422 can be attached to the right shoulder of the individual, and in embodiments, a third sensor 424 can be attached high and across the back of the individual.

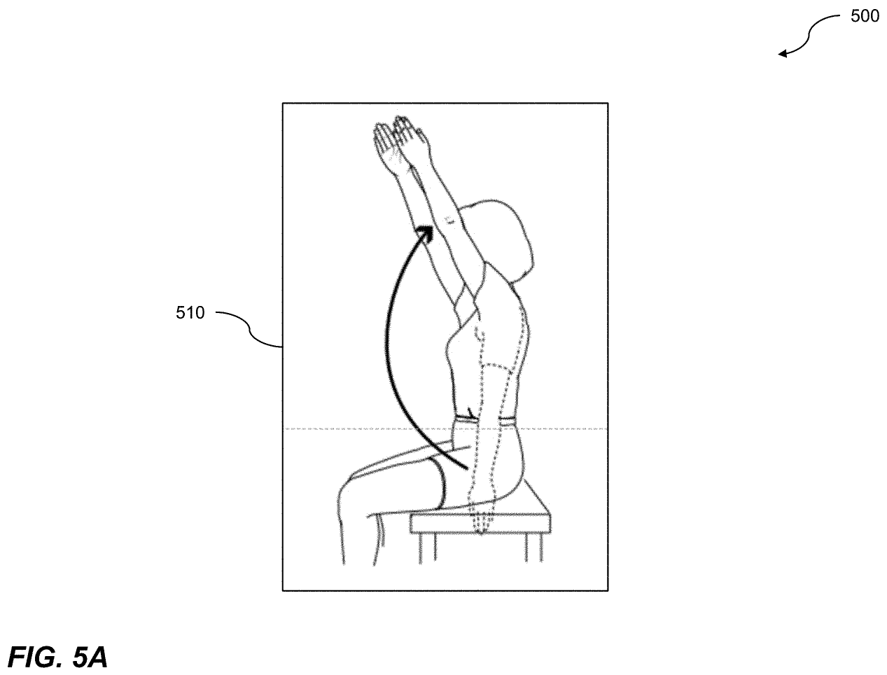

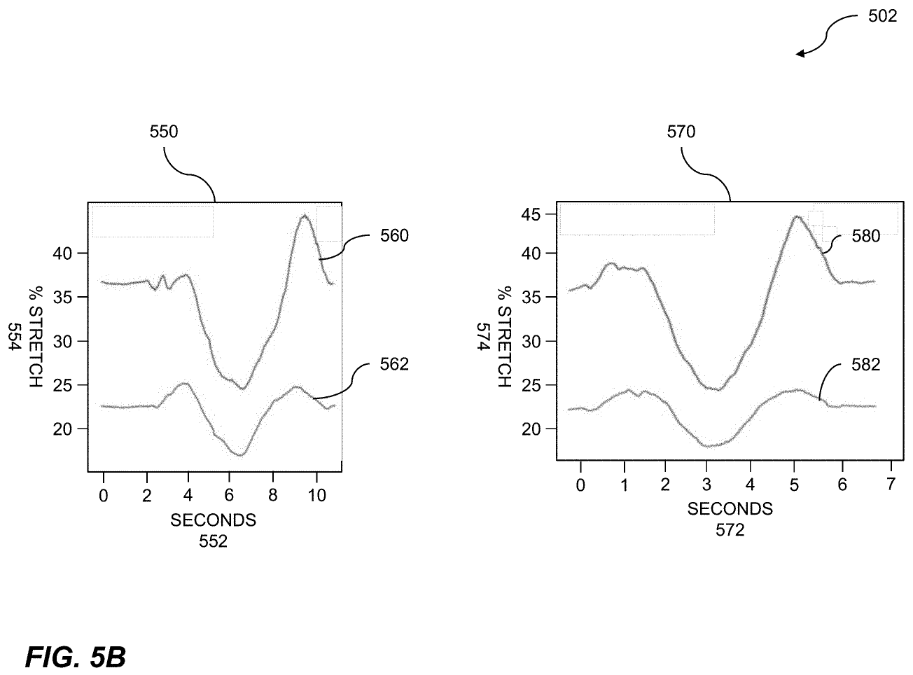

FIG. 5A shows shoulder motion. Two or more sensors such as stretch sensors and inertial measurement units (IMU) can be used for body part motion analysis using kinematics. The sensors are attached to a body part of an individual. The sensors enable collection of motion data of the body part. Sensor data is collected, where the sensors provide electrical information based on a micro-expression of movement of the body part. The electrical information is analyzed to generate a kinematic phase pattern. The kinematic phase pattern can be rendered on a visual display, where the kinematic phase pattern is rendered as part of a kinematic sequence. The visual display can include an animation of the body part.

FIG. 5A shows an example of shoulder motion 500. Two or more sensors can be attached to left and right shoulders of a person 510, where the person can be a patient, test subject, and so on. The sensors attached to the shoulders of the patient can be used to test for and quantify a severity and a location of a loss of joint stability. In the figure, the patient can raise or lower her left and right arms together as the motion of her left and right shoulders is measured. Embodiments include attaching at least a third sensor to the body part, where in this example, the body part is the shoulder region of the individual. The third sensor can be used to enable body part symmetry analysis. The body part symmetry analysis can be used to determine that two body parts, such as left shoulder and right shoulder, and moving properly. In embodiments, the at least third sensor enables an objective measurement of scapular movement.

FIG. 5B shows data collected from shoulders 502. The collected data can include electrical information from a stretch sensor, an IMU, and so on. Plot 550 can show flex return of the left scapula 562 and the flex return of the right scapula 560. The plot 550 can include a time scale in seconds 552 and a percentage stretch 554. The plot 550 shows that the percent stretch of the left scapula and the right scapula differ. The motion of the body part can be measured as the patient performs a similar arm lowering motion with the patient holding one or more weights. A weight can be held in each hand, the weight can be shared between the left hand and the right hand, etc. The plot 570 shows the flex return with weight of the left scapula 582 and the flex return with weight of the right scapula 580. The plot 570 can include a time scale in second 572 and a percentage stretch 574. Again, a difference in displacement between the left scapula and the right scapula is shown. For this example, less displacement of the scapula is preferred to more displacement. An increase in displacement of a scapula can indicate impairment.

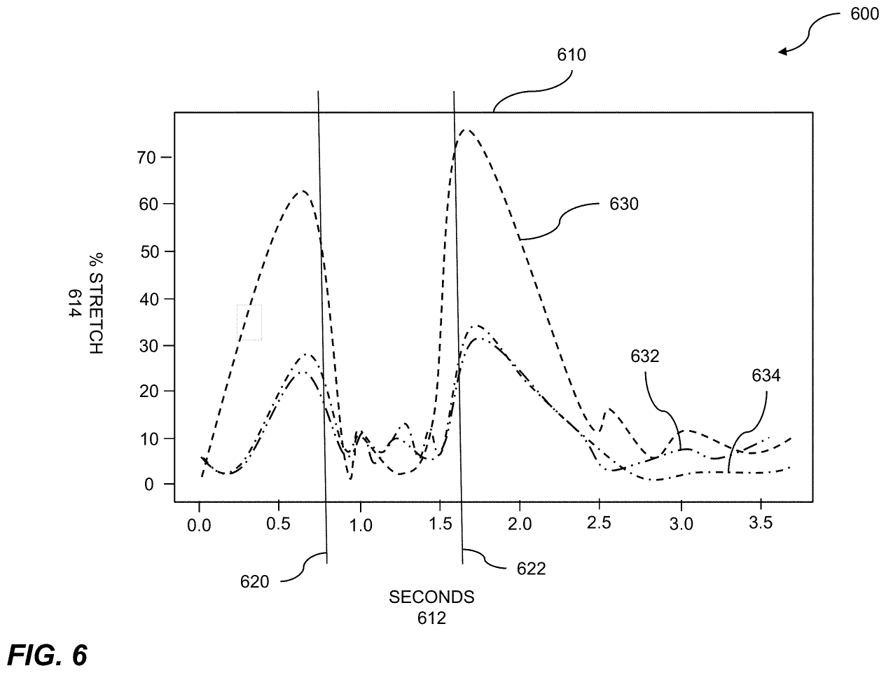

FIG. 6 shows a plot of jump data 600. Body part motion analysis uses kinematics. Two or more sensors such as a stretch sensor and an inertial measurement unit (IMU) are attached to a body part, where the sensors can enable collection of motion data of a body part. Data is collected from the sensors, where the sensors provide electrical information. The electrical information is based on micro-expression of body part movement. The electrical information is analyzed to generate a kinematic phase pattern. A plot 610 of jump data is shown. The two or more sensors can be attached to joints such as a hips, knees, or ankles. The sensors can be attached to muscles such as quadriceps, calf or gastrocnemius and soleus muscles, and so on. Data based on electrical information collected from stretch sensors or IMUs can be analyzed and plotted. The plot 610 can include a percentage stretch 614 versus time in seconds 612. The plot for the jump can show jump takeoff 620 and jump land 622. The takeoff can correspond in time to a maximum compression of left 632 or right 634 quadricep, and landing can similarly correspond in time to a maximum compression of left or right quadricep. The plot for deflection of or force on left knee 630 or right knee can also be shown. Changes in electrical characteristics by a stretch sensor or an IMU can be rendered along with an animation. The animation can include a human body, a body part of the human body, etc.

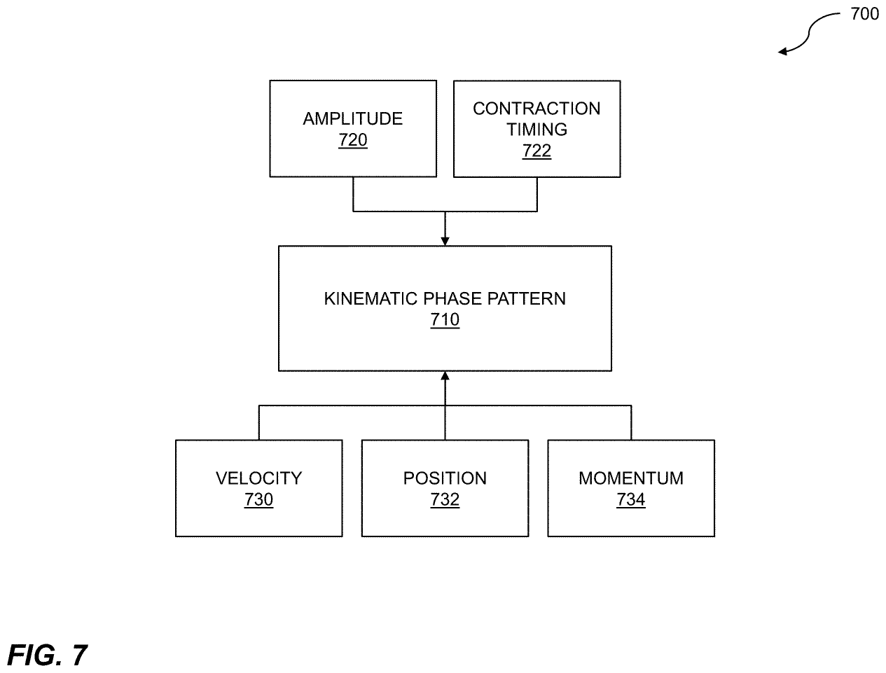

FIG. 7 shows a block diagram for a kinematic phase pattern from muscle data. Sensors, including two or more sensors attached to a body part of an individual, enable collection of motion data of the body part. The collected body part motion data enables body part motion analysis using kinematics. The sensors provide electrical information based on micro-expression of movement of the body part. The electrical information is analyzed to generate a kinematic phase pattern, which can be rendered as part of a kinematic sequence. The rendering can be displayed visually, where the display can include an animation of the body part. The sensors that provide the electrical information can include a stretch sensor, an accelerometer, a gyroscope, or a magnetometer.

The block diagram 700 includes a kinematic phase pattern 710. A kinetic phase pattern can include one or more of angles, positions, accelerations, and velocities of segments of body parts and joints during the motions of the body parts and joints. A kinematic phase pattern can refer to a cycle of movements such as movements related to walking or running, raising and lowering arms, and so on. A phase can include a stance-phase. A stand-phase can include a double stance from walking. The double stance can include a heel strike, a mid-stance where the legs are vertical, a toe-off, and so on. The double stance can include a movement of a center of mass. In the case of walking, an individual's head will appear to rise at mid-stand and fall for heel-strike or toe-off. Another phase can include a swing-phase. In a swing phase, a leg to be moved forward undergoes knee-flexion before being swung forward.

In order to form a kinematic phase pattern, various types of information can be collected. The types of information can be based on micro-expressions of movement of the body part of the individual. In embodiments, the micro-expression of movement of the body part includes muscle contraction amplitude 720 and muscle contraction timing 722. The muscle contraction amplitude and the muscle contraction timing can be collected from one or more sensors such as stretch sensors. In other embodiments, the micro-expression of movement of the body part can include linear movements and rotational movements. Linear movements can include raising or lowering arms or legs, while rotational movements can include clockwise or anticlockwise rotations of shoulders, elbows, wrists, hips, knees, or ankles. In embodiments, the linear movements and the rotational movements each can comprise velocity 730, position 732, and momentum 734. The velocity, position, and momentum can be collected from one or more sensors such as inertial measurement units (IMU). In embodiments, the velocity, position, and momentum each can include a magnitude and a time-dependent function. The velocity, position, or momentum of the body part change as the individual moves that body part. Various coordinate systems can be used to describe micro-expressions of movement of the body part. In embodiments, the position can comprise a three-dimensional coordinate. Momentum can also be referenced using a variety of techniques. In embodiments, the momentum can include a center mass of a segment of a body part.

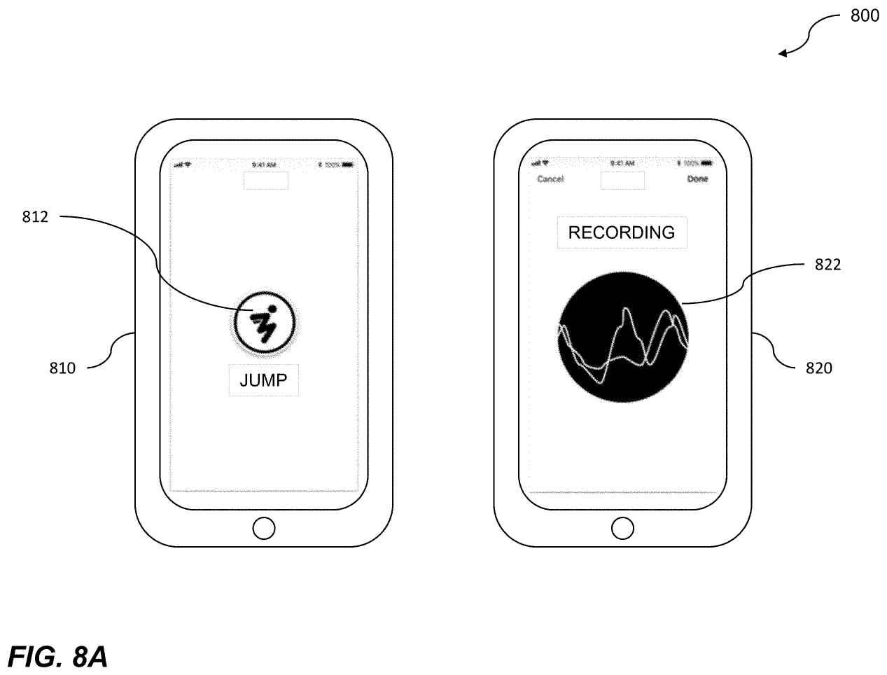

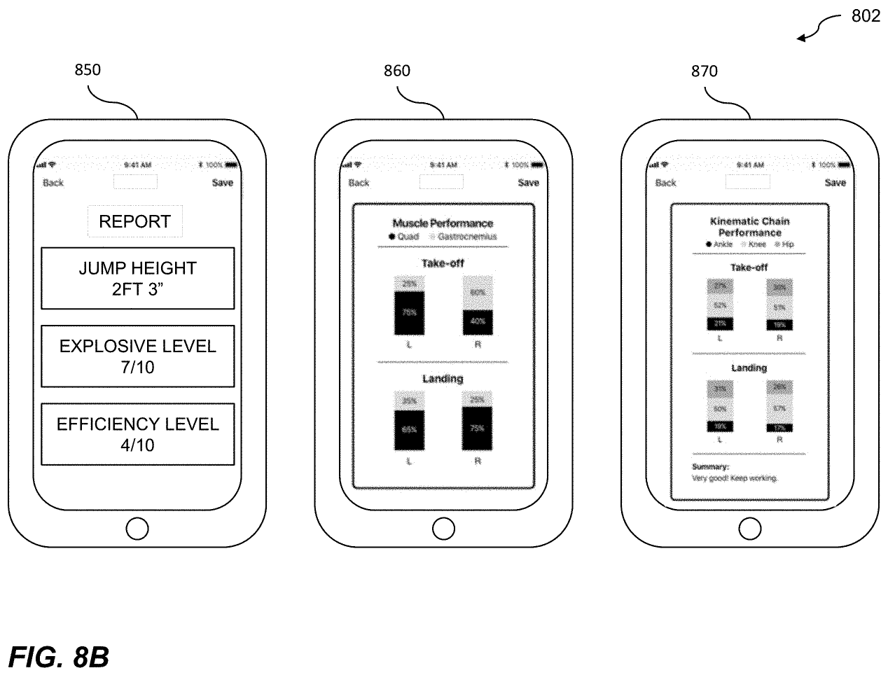

FIG. 8A illustrates a dashboard showing jump and recording. As discussed throughout, one or more wearable sensors can be used to collect data relating to a body part of an individual. The data that is collected can include electrical information from the one or more sensors. The body part can include a muscle, a joint, and so on. The collected electrical information can be analyzed to generate a kinetic phase pattern. The kinematic phase pattern can be rendered as part of a kinematic sequence. A dashboard is shown for jump 800. The dashboard can be rendered on a screen such as a screen coupled to an electronic device 810 including a smartphone, a tablet, a PDA, a laptop, and so on. The dashboard can include one or more icons, where the icons can show a variety of tests, exercises, therapies, etc., that can be undertaken by an individual. The dashboard can include one or more buttons such as a jump button 812. Touching or clicking the button can initiate an operation such as collecting information from one or more sensors attached to the individual.

Once a button has been touched or clicked, an operation related to the button can be initiated. The dashboard can be displayed on the same electronic device 810 or a different electronic device 820. In embodiments, the electronic devices 810 and 820 can be the same electronic device. The operation related to the button press can be displayed. The operation can be a real-time operation where collected information such as electrical information from the one or more sensors can be rendered within the dashboard. The rendering can be a visual display such as a graph, an animation, a varying color, a changing audio signal, and the like. In embodiments, the rendering 822 on electronic device 820 shows information related to the individual executing a jump.

FIG. 8B illustrates a dashboard showing a jump report 802. Information such as electrical information collected from one or more sensors attached to a body part of an individual can be analyzed and rendered on an electronic display. The rendering on the electronic display can include a report, where the report can include multiple panes or views that can be accessible through a dashboard. The electronic display can be coupled to an electronic device such as a smartphone, tablet, PDA, laptop computer, and the like. The report can be based on sensor information collected and analyzed for a motion of the body part. The motion of the body part can be related to a research study, an evaluation of the patient, an exercise, a therapy such as physical therapy, and so on.

Three example panes or views of a dashboard showing a jump report are shown, 850, 860, and 870. The view 850 can include data relating to a jump or other movement executed by the individual. The report can include analysis data such as jump height, explosive level, efficiency level, and so on. The analysis data can be computed based on MKS units, English units, etc. The analysis data can be determined based on an average performance, a desired performance level, etc. The average performance, for example, can be based on an average performance for the individual, an average performance based on a similar demographic group, and the like. The view 860 can include muscle performance. The muscle performance can include the performance of symmetrical muscles or muscle groups. Symmetrical muscles can include left quadricep and right quadricep; left bicep and right bicep; left triceps and right triceps; etc. The muscle performance of the symmetrical muscles can show whether each side is performing normally, one side is weaker than the other side, an indication or risk of injury is evident, and so on. The muscle performance for a maneuver such as the jump can include data related to a takeoff or a landing. The view 870 can include kinetic chain performance. The kinetic chain performance can include data relating to adjacent joints. In the case of a jump, the kinetic chain performance can include information relating to the individual's left hip and right hip; left knee and right knee, and left ankle and right ankle. The kinematic chain performance can further include information relating to the jump takeoff and jump landing, as described previously.

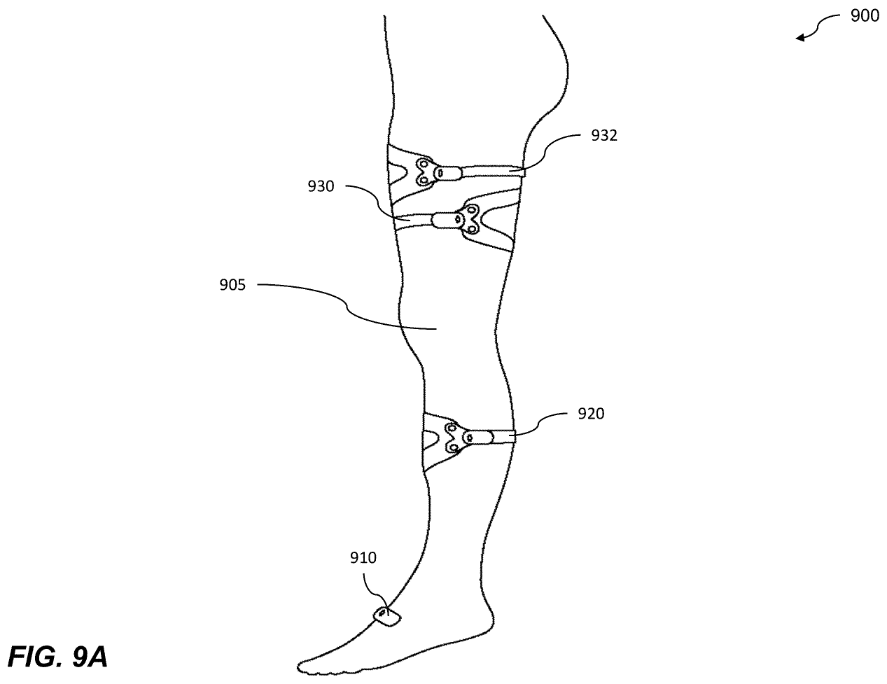

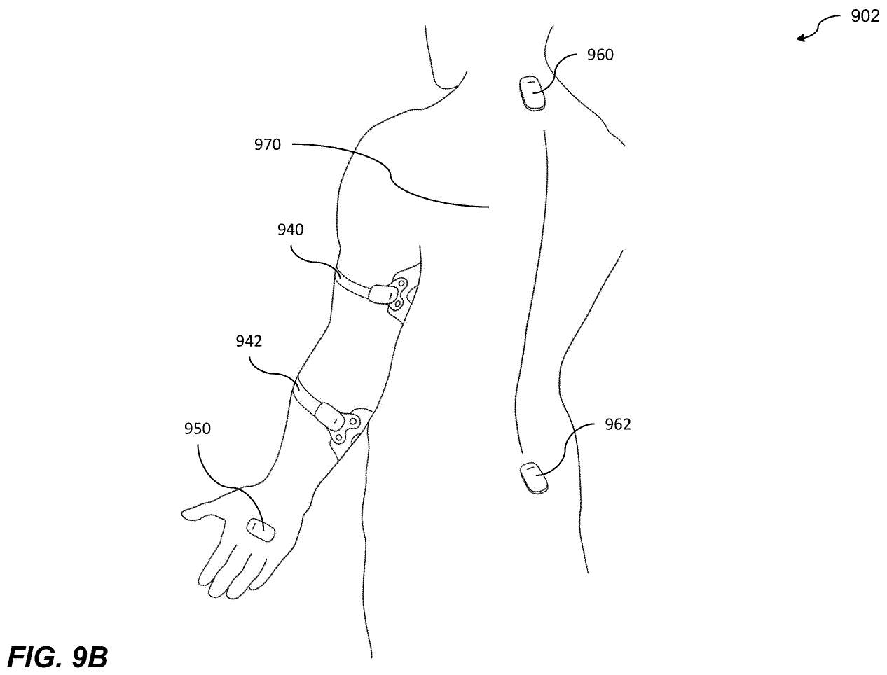

FIG. 9A shows example lower body sensor locations 900. As discussed throughout, one or more wearable sensors can be used to collect data relating to a body part of an individual. The data that is collected can include electrical information from the one or more sensors. The body part can include a muscle, a joint, and so on. The collected electrical information can be analyzed to generate a kinetic phase pattern. The kinematic phase pattern can be rendered as part of a kinematic sequence. A lower human body 905 to which sensors of various types can be mounted is shown. The sensors can include IMUs, muscle activity sensors, linear displacement or stretch sensors, and so on. The sensor can be mounted to the human body at various locations and for a variety of purposes. The body parts or locations can include individual body parts such as an arm, shoulder, hip, knee, leg, etc. The body parts or locations can include symmetric locations such as left and right shoulder, elbow, hip, or knee; left and right arm or leg; and the like. The sensors which can be mounted on the human body can include single-type sensors such as IMU, muscle activity, or linear displacement sensors; or can include combination sensors that can comprise two or more types of sensors. In embodiments, the “combination” sensors can include IMU and muscle activation sensors.

In embodiments, sensors can be applied to one leg or both legs. In the figure, sensors 930 and 932 are applied to the thigh of a leg, and sensor 920 can be applied to the calf of the leg. Sensors 920, 930, and 932 can be combination sensors that include both IMUs and muscle activation sensor. In addition, sensor 910 can be applied to the top of the foot of lower body 905. Sensor 910 can be only an IMU sensor, which can provide baseline lower body positioning data and the like.

FIG. 9B shows example upper body sensor locations 902. As discussed throughout, one or more wearable sensors can be used to collect data relating to a body part of an individual. The data that is collected can include electrical information from the one or more sensors. The body part can include a muscle, a joint, and so on. The collected electrical information can be analyzed to generate a kinetic phase pattern. The kinematic phase pattern can be rendered as part of a kinematic sequence. An upper human body 970 to which sensors of various types can be mounted is shown. The sensors can include IMUs, muscle activity or activation sensors, linear displacement or stretch sensors, and so on. The sensor can be mounted to the human body at various locations and for a variety of purposes. The body parts or locations can include individual body parts such as an arm, shoulder, hip, knee, leg, etc. The body parts or locations can include symmetric locations such as left and right shoulder, elbow, hip, or knee; left and right arm or leg; and the like. The sensors which can be mounted on the human body can include single-type sensors such as IMU, muscle activity, or linear displacement sensors; or can include combination sensors that can comprise two or more types of sensors. In embodiments, the “combination” sensors can include IMU and muscle activation sensors.

In embodiments, sensors can be mounted at specific locations on the upper human body such as at the neck 960, at the small of the back 962, and so on. Sensor 960 and sensor 962 can include IMUs. Sensors 960, 962, and others (not shown) can be particularly useful for detecting, calculating, or determining body motions. Other sensors can be applied to various body parts. In embodiments, sensors can be applied to one arm or both arms. In the figure, sensor 940 is applied to an upper arm, and sensor 942 is applied to the lower arm. The arm sensors can include IMUs and/or muscle activity sensors. The arm sensors can be the same types of sensors or may include different types of sensors, such as IMU sensor 950 on the back of a hand, which can be an IMU sensor to determine baseline hand/arm movement and positioning. When sensors are applied to both arms, movement, muscle displacement, etc., can be compared between the arms. Such comparisons are useful for detecting imbalances or asymmetries between muscles of the limbs, gauging recovery from injury, etc.

In other embodiments, not shown, other numbers of wearable sensors may be applied to the various body parts. The wearable sensors can include muscle activation sensors, skeletal movement sensors, linear displacement sensors, inertial measurement unit sensors, and so on. The sensors can be applied to body parts in order to measure muscle or joint activity, displacement, deformation, etc. A sensor can be applied to the knee, for example. The sensor can be used to measure motion of the knee such as a number of degrees of flexion of the knee as the person to whom the sensor is applied engages in various activities such as standing, walking, running, bicycling, dancing, swimming, and so on. Other sensors may be applied to the leg. A sensor can be applied to a quadricep. A sensor can be applied to a calf. The data collected or obtained from the sensors may be aggregated. Sensors can be applied to one leg or both legs, one arm or both arms, one scapula or both scapula, etc. When sensors are applied to both legs or other limbs, for example, the data collected from the sensors of the left limb and the sensors of the right limb can be analyzed for symmetry such as body posture symmetry. The data obtained from the sensors may also be used to quantify differences in muscle activity, joint movement, etc. The quantified difference can be correlated with changes in footwear, protective gear, clothing, and so on.

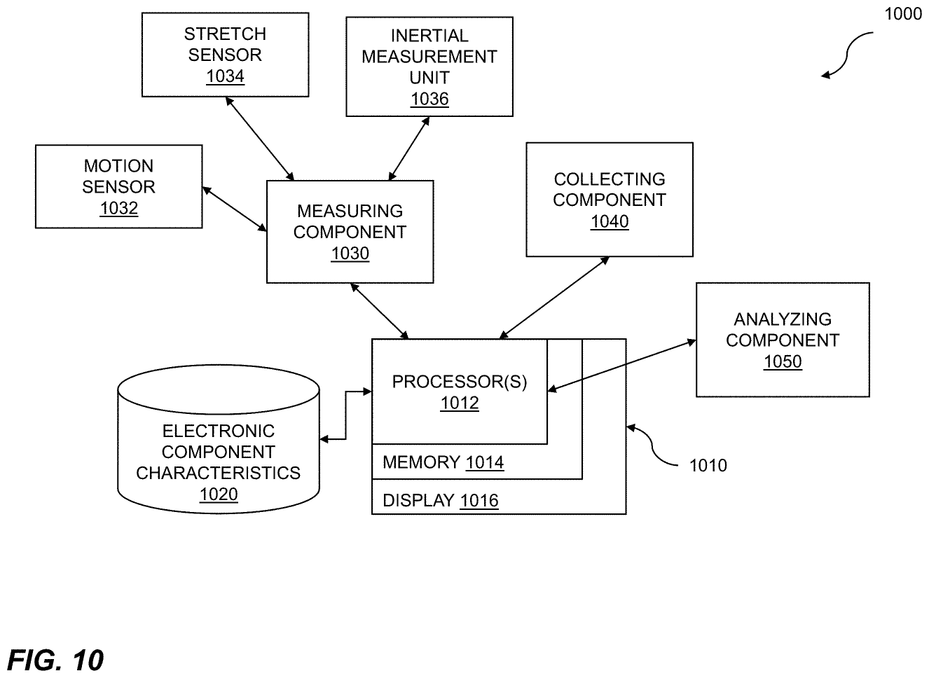

FIG. 10 is a system diagram for body part motion analysis using kinematics. Kinematic analysis can describe motion of a body part including a joint such as a shoulder, elbow, hip, knee, ankle, wrist, and so on. Sensors, including body-attachable sensors, can be used to analyze motion of a body part. Sensors can be applied to a body part of an individual, where the application can be accomplished using tape or straps using hooks, suction cups, wraps, and so on. The sensors can include stretch sensors or inertial measurement units (IMU). The IMUS can include an accelerometer, a gyroscope, a magnetometer, etc. The stretch sensor or the IMU can change electrical characteristics, where the electrical characteristics can change based on the sensor stretching, accelerating, moving, and the like. The electrical characteristics can include electrical information, where the electrical information can be based on micro-expressions of movement of the body part. Processors can be used to analyze the electrical information collected from the sensors. The collected electrical information can be used to generate a kinematic phase pattern. The kinematic phase pattern can be rendered as part of a kinematic sequence, where the rendering can be displayed visually. In embodiments, the rendering includes an animation of the body part. The data relating to the motion of the body part can be used for body part treatment including medical techniques, physical therapy, occupational therapy, athletic training, strengthening, flexibility, endurance, conditioning, or rehabilitation therapy treatment.

The system 1000 can include an analysis computer 1010. The analysis computer can include one or more processors used to analyze electrical information from two or more sensors. The analysis performed by the analysis computer can generate a kinematic phase pattern. The analysis computer 1010 can comprise one or more processors 1012, a memory 1014 coupled to the one or more processors 1012, and a display 1016. The display 1016 can be configured and disposed to present collected data, analysis, intermediate analysis steps, instructions, algorithms, or heuristics, and so on. In embodiments, one or more processors are attached to the memory, where the one or more processors, when executing the instructions which are stored, are configured to: attach two or more sensors to a body part of an individual, wherein the two or more sensors enable collection of motion data of the body part; collect data from the two or more sensors, wherein the two or more sensors provide electrical information based on a micro-expression of movement of the body part; and analyze the electrical information from the two or more sensors to generate a kinematic phase pattern.

The system 1000 can include an electronic component characteristics component 1020. The electronic component characteristics can include a library of lookup tables, resistance characteristics, capacitance characteristics, functions, algorithms, routines, and so on, that can be used for the analysis of the electrical information collected from the one or more sensors. In a usage example, the electrical component characteristics can include a lookup table that enables mapping of an electrical signal from a stretch sensor to millimeters of motion of the body part. The system 1000 can include a measuring component 1030. The measuring component can act as an interface between one or more sensors and the analysis computer 1010. The measuring component can measure electrical signals, where the electrical signals can be generated by a motion sensor 1032, a stretch sensor 1034, an inertial measurement unit 1036, etc.

The system 1000 can include a collecting component 1040. The collecting component 1040 can include resistance and/or capacitance measuring hardware, and can include hardware for measuring current, voltage, resistance, capacitance, impedance, and/or inductance. A generating component (not shown) can include hardware for generating direct current and/or alternating current signals used for obtaining resistance and/or capacitance measurements. Typically, the current values are low (e.g. microamperes) and in embodiments, the frequency range includes signals from about 100 hertz to about 1 megahertz. The system 1000 can include an analyzing component 1050. The analyzing component can analyze the electrical information that is collected from various sensors such as stretch sensors, inertial measurement units, and so on. The analysis can be performed on electrical signals related to stretch, acceleration, rotational motion, magnetic field, and the like.

The system 1000 can include a computer program product embodied in a non-transitory computer readable medium for motion analysis, the computer program product comprising code which causes one or more processors to perform operations of: attaching two or more sensors to a body part of an individual, wherein the two or more sensors enable collection of motion data of the body part; collecting data from the two or more sensors, wherein the two or more sensors provide electrical information based on a micro-expression of movement of the body part; and analyzing, using one or more processors, the electrical information from the two or more sensors to generate a kinematic phase pattern.

The system 1000 can comprise a computer system for motion analysis comprising: a memory which stores instructions; one or more processors attached to the memory wherein the one or more processors, when executing the instructions which are stored, are configured to: attach two or more sensors to a body part of an individual, wherein the two or more sensors enable collection of motion data of the body part; collect data from the two or more sensors, wherein the two or more sensors provide electrical information based on a micro-expression of movement of the body part; and analyze the electrical information from the two or more sensors to generate a kinematic phase pattern.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for motion analysis comprising:
attaching two or more sensors to a body part of an individual, wherein the two or more sensors enable collection of motion data of the body part, and wherein the two or more sensors include at least one inertial measurement unit (IMU) and at least one sensor determining muscle activation;
collecting data from the two or more sensors, wherein the two or more sensors provide electrical information based on a micro-expression of movement of the body part; and
analyzing, using one or more processors, the electrical information from the two or more sensors to generate a kinematic phase pattern.

2. The method of claim 1 wherein the two or more sensors comprise one or more integrated sensors.

3. The method of claim 2 wherein the one or more integrated sensors comprise stretch sensors.

4. (canceled)

5. The method of claim 1 wherein the two or more sensors capture two or more modalities of body part motion.

6. The method of claim 1 wherein at least one of the two or more sensors comprises a bending sensor.

7. The method of claim 1 wherein at least one of the two or more sensors comprises a mechanomyogram sensor.

8. The method of claim 1 wherein at least one of the two or more sensors comprises an electromyogram sensor.

9. (canceled)

10. The method of claim 1 further comprising rendering the kinematic phase pattern as part of a kinematic sequence.

11. The method of claim 10 wherein the rendering enables additional kinematic analysis.

12-13. (canceled)

14. The method of claim 1 wherein the micro-expression of movement of the body part includes linear movements and rotational movements.

15. The method of claim 14 wherein the linear movements and the rotational movements each comprises velocity, position, and momentum.

16. The method of claim 15 wherein the velocity, position, and momentum each comprises a magnitude and a time-dependent function.

17. The method of claim 15 wherein the position comprises a three-dimensional coordinate.

18. The method of claim 15 wherein the momentum includes a center mass of a segment of a body part.

19. The method of claim 1 wherein the micro-expression of movement of the body part includes muscle contraction amplitude and muscle contraction timing.

20. The method of claim 1 further comprising calculating a kinematic summation and distribution ratio based on the micro-expression of movement of the body part.

21. The method of claim 20 wherein the calculating provides information on kinematic phases.

22. The method of claim 21 further comprising using the information on kinematic phases to build a kinematic phase library.

23. The method of claim 22 further comprising using the kinematic phase library to enable pattern recognition for information on kinematic phases obtained from the calculating.

24. The method of claim 20 further comprising combining the kinematic summation and distribution ratio with one or more additional kinematic summation and distribution ratios for a segment of a related body part.

25. The method of claim 24 wherein the combining describes a kinematic sequence.

26. The method of claim 24 wherein the combining enables micro-expression analysis of the individual.

27-31. (canceled)

32. The method of claim 1 further comprising attaching at least a third sensor to the body part.

33. The method of claim 32 wherein the at least a third sensor enables body part symmetry analysis.

34-35. (canceled)

36. A computer program product embodied in a non-transitory computer readable medium for motion analysis, the computer program product comprising code which causes one or more processors to perform operations of:
attaching two or more sensors to a body part of an individual, wherein the two or more sensors enable collection of motion data of the body part;
collecting data from the two or more sensors, wherein the two or more sensors provide electrical information based on a micro-expression of movement of the body part; and
analyzing, using one or more processors, the electrical information from the two or more sensors to generate a kinematic phase pattern.

37. A computer system for motion analysis comprising:
a memory which stores instructions;
one or more processors attached to the memory wherein the one or more processors, when executing the instructions which are stored, are configured to:
attach two or more sensors to a body part of an individual, wherein the two or more sensors enable collection of motion data of the body part;
collect data from the two or more sensors, wherein the two or more sensors provide electrical information based on a micro-expression of movement of the body part; and
analyze the electrical information from the two or more sensors to generate a kinematic phase pattern.
